(19) United States
(12) Patent Application Publication
(10) Pub. No.: US 2026/0183085 A1
(43) Pub. Date: Jul. 2, 2026

(54) METHOD AND SYSTEM FOR TEETH SEGMENTATION AND/OR GENERATING ORTHODONTIC AND/OR DENTAL APPLIANCES

(71) Applicants: Pramod PHILIP, Aurora, CO (US); Sarah ANVERY, New York, NY (US)

(72) Inventors: Pramod PHILIP, Aurora, CO (US); Sarah ANVERY, New York, NY (US)

(21) Appl. No.: 19/130,288

(22) PCT Filed: Nov. 16, 2023
(86) PCT No.: PCT/US2023/079998

§ 371 Date: May 15, 2025

Related U.S. Application Data
(60) Provisional application No. 63/425,838, filed on Nov. 16, 2022.

(51) Int. Cl. A61C 7/00; A61C 7/08

(52) U.S. Cl. CPC A61C 7/002; A61C 7/006; A61C 7/08

(57) ABSTRACT

The present exemplary embodiments relate to a method and system for virtual segmentation of teeth and jaw and/or generating orthodontic appliances, using 3D models on a computer, tablet, or any similar device. It finds particular application in conjunction with magnetic orthodontic appliances or aligners, and will be described with particular reference thereto. However, it is to be appreciated that the present exemplary embodiments are also amenable to other applications, for example, where improved method of virtual segmentation, treatment simulation and/or appliance fabrication is desired in a variety of other environments.

The present application is based on and claims priority to U.S. Provisional Application Ser. No. 63/425,838 filed Nov. 16, 2022, which is incorporated herein by reference in its entirety.

### TECHNICAL FIELD

The present exemplary embodiments relate to a method and system for virtual segmentation of teeth and jaw and/or generating orthodontic appliances, using 3D models on a computer, tablet, or any similar device. It finds particular application in conjunction with magnetic orthodontic appliances or aligners, and will be described with particular reference thereto. However, it is to be appreciated that the present exemplary embodiments are also amenable to other applications, for example, where improved method of virtual segmentation, treatment simulation and/or appliance fabrication is desired in a variety of other environments.

### BACKGROUND

Orthodontic tooth movements were carried out with the help of traditional braces attached on the labial or outer visible aspect of the teeth or lingual or the back surface of the teeth facing the tongue. Advancement in the digital technology has allowed clinicians to virtually plan the various teeth movements and fabricate appliances before making it a physical reality. The basis of all these technologies is the possibility to obtain a 3D virtual model of the teeth and the supporting structures using an intra oral scan and the availability of software that facilitates virtual segmentation of the teeth followed by movement of the teeth in all three planes of space. The possibility of obtaining a 3D virtual model of the facial skeleton and the teeth from 3D Cone Beam Computed Tomographic images (3D radiograph) has facilitated the simulation of surgical movements of the jaws while correcting discrepancies in the position, size or orientation of the jaws. It also allows the placement of different attachments while employing clear aligner therapy, with various attachments obtained from a library. This approach has been used for staging orthodontic tooth movement using various clear aligners and some of the lingual appliances.

Also, the typical software employed by various companies for segmentation and/or modeling are operated with the help of mouse and/or track pad of the computer. Although it has been very popular, it is not very user friendly due to the following reasons. Orientation of the models, identification of the tooth/teeth margins and boundaries, identification of the long axis of the tooth/teeth etc. are dependent on precise identification of the respective lines/landmarks using a mouse. Moreover, the planned teeth movement in all three planes of space is simulated on the virtual model using tabs for rotational or translational movements. Rotational movements in various software were achieved by turning a wheel on the monitor (with the help of mouse or trackpad) that represents the direction of rotation with an axis planned for deciding the center of rotation whereas translational movements were achieved by tabs for the same in different axis of directions. Also, some software uses the incremental movements in degree and millimeter by direct input of precise numbers to achieve jaw and/or tooth movement.

Similar software is used in restorative dentistry for planning restorations of missing tooth structure or the whole tooth/teeth, planning of removable and fixed dentures and implant placement.

Although precision is quite good with various software, these are not user friendly as these minute movements are carried out with the help of a computer mouse/trackpad which are not originally designed for the same.

### BRIEF DESCRIPTION

In one aspect of the presently described embodiments, a system for generating a magnetic based orthodontic appliance includes at least one processor, at least one memory having code or instructions stored thereon that, when executed by the at least one processor, causes the system to receive information on a patent's teeth including at least one of photographic images of teeth and/or face, radiographs, virtual models, and a treatment plan for generating the magnet based orthodontic appliance, virtually segment the teeth, virtually determine attachments for target teeth and support teeth, selectively virtually modify the attachments, selectively virtually alter the orientation of the attachments, create a virtual meshwork on surfaces of the attachments, virtually fabricate support arches with corresponding virtual attachments, output a virtual magnetic based orthodontic appliance.

In another aspect of the presently described embodiments, the system includes attachments for target teeth that are magnets or paramagnets.

In another aspect of the presently described embodiments, the system includes attachments for support teeth that are magnets or paramagnets.

In another aspect of the presently described embodiments, the system includes attachments for support arches that are magnets or paramagnets.

In another aspect of the presently described embodiments, a method for generating a magnetic based orthodontic appliance includes virtually segmenting the teeth, virtually determining attachments for target teeth and support teeth, selectively virtually modifying the attachments, selectively virtually altering the orientation of the attachments, creating a virtual meshwork on surfaces of the attachments, virtually fabricating support arches with corresponding virtual attachments, outputting a virtual magnetic based orthodontic appliance.

In another aspect of the presently described embodiments, the method includes attachments for target teeth that are magnets or paramagnets.

In another aspect of the presently described embodiments, the method includes attachments for support teeth that are magnets or paramagnets.

In another aspect of the presently described embodiments, the method includes attachments for support arches that are magnets or paramagnets.

In another aspect of the presently described embodiments, a system for segmenting teeth and/or generating an orthodontic appliance includes at least one processor, at least one memory having code or instructions stored thereon that, when executed by the at least one processor, cause the system to receive information on a patient's teeth including at least one of photographic images, radiographs, virtual models, and a treatment plan for the patient's teeth, and virtually segment the teeth using a touch screen receptive for single or multi touch based commands using fingers or a stylus on the touch screen, gestures or by using voice control, to virtually move the teeth.

In another aspect of the presently described embodiments, the system further includes virtually fabricating an orthodontic appliance.

In another aspect of the presently described embodiments, the orthodontic appliance is at least one of: an aligner or a magnetic based orthodontic appliance.

In another aspect of the presently described embodiments, a method for segmenting teeth and/or generating an orthodontic appliance includes receiving information on a patient's teeth including at least one of photographic images, radiographs, virtual models, and a treatment plan for the patient's teeth, and virtually segmenting the teeth using a touch screen receptive for single or multi touch based commands using fingers or a stylus on the touch screen, gestures or by using voice control, to virtually move the teeth.

In another aspect of the presently described embodiments, the method includes fabricating an orthodontic appliance.

In another aspect of the presently described embodiments, the orthodontic appliance is at least: an aligner or a magnetic based orthodontic appliance.

### BRIEF DESCRIPTION OF THE DRAWINGS

FIG. 1 is a flowchart of an example method(s) according to the presently described embodiments;

FIG. 2 is an illustration according to the presently described embodiments;

FIG. 3 is an illustration according to the presently described embodiments;

FIG. 4 is an illustration according to the presently described embodiments;

FIG. 5 is an illustration according to the presently described embodiments;

FIG. 6 is an illustration according to the presently described embodiments;

FIG. 7 is a flowchart of an example method(s) according to the presently described embodiments;

FIG. 8 is an example system according to the presently described embodiments; and,

FIGS. 9A-9C show an example of a magnet based orthodontic appliance.

### DETAILED DESCRIPTION

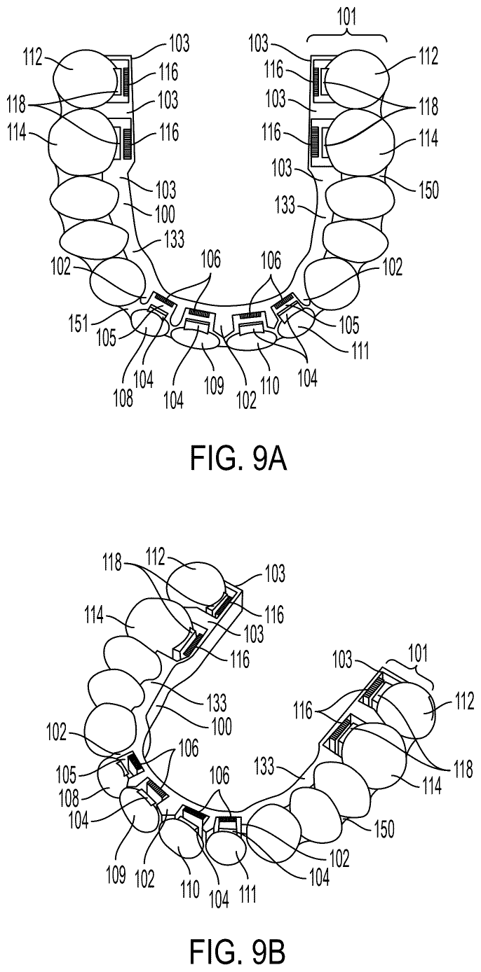

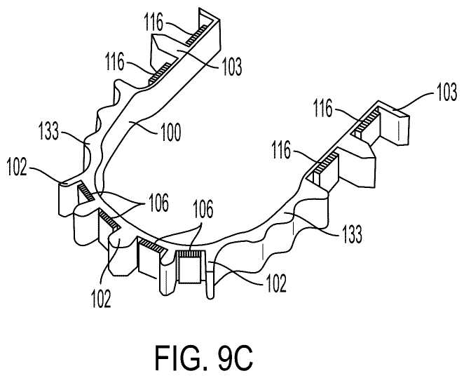

Magnet based orthodontic appliances are one of the recent developments which uses magnetic force to move teeth into desired position. One example of such a system and/or method is described in a patent application entitled, “Magnet-Based Orthodontic Appliance,” having U.S. application Ser. No. 17/744,037, filed on May 13, 2022 (and published as U.S. Publication No. 2022/0361984A1, which is hereby incorporated by reference herein in its entirety. This noted prior application shows several embodiments of a magnet based orthodontic appliance. These structures can be generated using the techniques of the presently described embodiments. In this regard, any of the embodiments could be generated; however, for brevity and conciseness, reference to FIGS. 9A-9C will suffice to provide context for terminology used herein.

As such, in an embodiment illustrated in FIGS. 9A-9C, a magnet-based orthodontic appliance system 101 includes at least one first docking component such as at least one attachment magnet 118 configured to be attached to the lingual or palatal side of at least one respective support tooth (e.g., 112, 114). For instance, four support teeth 112 and 114 might be used. The magnet based orthodontic appliance system 101 includes a rigid support arch 100 having respective at least one second docking component such as at least one mounting magnet 116. The appliance system can include stops. For example, the support arch 100 includes one or more stops in the form of stop arch extensions 103. The rigid support arch 100 is secured in position by the docking components, for example, by attractive magnetic forces between the mounting magnets 116 and attachment magnets 118. Though there is an attractive force between the mounting magnets 116 and attachment magnets 118, which might otherwise be carried through to the support teeth 112, 114, the support teeth 112, 114 are protected from those forces and are prevented from significant movement toward the rigid support arch 100 by stops (e.g., 103), which oppose the attractive force by physically preventing tooth movement. In the illustrated embodiment, stops or arch extensions 103 are configured to contact the lingual or palatal side of the support teeth 112, 114 and maintain a separation between the first docking component(s), in this case attachment magnets 118 and the second docking component(s), in this case the mounting magnets 116. The rigid support arch 100 is configured to be held in place by magnetic forces between the attachment magnets 118 and mounting magnets 116. Stop arch extensions 103 prevent direct contact of the approximating surfaces. Close approximation 133 of the rigid support arch 100 to the palatal or lingual surface of other teeth of the illustrated set of teeth 150 can be used to provide a guide for the fit of the appliance in the mouth when the patient is inserting it. The rigid support arch 100 supports at least one therapeutic magnet 106 at therapeutic positions corresponding to at least one target magnet 104 attached to the palatal or lingual side of at least one target tooth 108-111. For instance, in the illustrated scenario, four target teeth 108-111 are mispositioned. For example, the teeth 108 111 have gaps or undesired space on either side. Respective pairs of the therapeutic magnets 106 and target magnets 104 would provide respective magnetic forces for urging (e.g., pulling) the target teeth 108-111 toward a preferred position. The appliance system 101 includes further stops in the form stop arch extensions 102 which extend from the rigid support arch 100 toward the target teeth 108-111. Initially the stops (extensions 102) do not make contact with the target teeth 108-111. The attractive force between the therapeutic magnets 106 and target magnets 104 pulls the target teeth 108-111 toward the rigid support arch 100 in order to urge the target teeth backward and reduce the size of the gaps or spaces between the target teeth and the teeth adjacent thereto. A space 105 between the therapeutic magnets 106 and the target magnets 104 is more than a space 151 between the extensions 102 from the support arch 100 and the palatal or lingual side of the target teeth 108-111. Accordingly, as the teeth are pulled back toward the desired position by the therapeutic forces between the therapeutic magnets 106 and the target magnets 104, the stops or extensions 102 would prevent direct contact between the therapeutic magnets 106 and the target magnets 104 and prevent the target teeth from being repositioned back further than desired. While direct contact may, in some scenarios be necessary or desirable, direct contact between the magnets on the teeth and those on the support arch would makes it difficult for the patient to insert and remove the appliance due to, for example, the very strong magnetic forces produced under those circumstances. Accordingly, another reason this embodiment includes the stops or extensions 102 is to prevent such direct approximation.

It is noted that while the word “magnet” is used herein throughout for clarity, it is to be understood that as used herein, the word “magnet” does not necessarily refer to a permanent magnet. In some embodiments or scenarios, it may be acceptable or beneficial for only one magnet of a magnet pair (e.g., a therapeutic and target magnet or an attachment and mounting magnet) to be a permanent magnet. The other magnet of a magnet pair may be a paramagnet or element made of material that becomes magnetized in the presence of a magnetic field. For example, one element of a pair might be made of stainless steel or other ferromagnetic material that becomes magnetized and drawn to its mate in the presence of the magnetic field from that mate. For simplicity and clarity, it is to be understood that in any description where attractive magnetic forces are discussed or implied, one of the magnets of the pair may be a paramagnetic element. In applications where repulsive forces are employed, both magnets must be permanent magnets so that like magnetic poles of the pair can be configured to face one another, thereby providing the repulsive force. Biocompatibility can be achieved by making the magnets of a bio-compatible material or by coating or encapsulating the magnets in a bio-compatible material. For example, the bio-compatible material that could be used to coat the magnet could be a varnish, plastic, gold, silver, steel, metal alloy, or other appropriate metal or metal alloy. In addition, a coating such as a gold coating could then be coated with a second coating such as steel or other appropriate bio-compatible material. The second coating such as steel could serve multiple purposes such as protecting the underlying coating and of enhancing the magnetic forces.

It should also be appreciated that the respective magnets for teeth may be attached to or incorporated into the teeth in a variety of manners including, for example, with use of cement or adhesives. Likewise, the respective magnets for the appliance may be unitary with the appliance (e.g., by stamping, casting, or additive manufacturing) or attached using any of a variety of manners including, for example, using screws, brackets, cement, adhesives, etc.

It should be appreciated that the example appliance illustrated in FIGS. 9A-9C is, again, merely an example of a magnet based orthodontic appliance that are produced by the presently described embodiments. The configurations shown in FIGS. 9A-9C will vary as a function of a variety of factors such as, but not limited to, the desired support arch, the desired magnetic material, and/or the desired tooth movement. The configuration of the support arch will also vary based on the number of teeth (including support and target teeth), the position of the teeth (including support and target teeth), and orthodontic technique or procedure being used to address the specific orthodontic issue. The configuration, purpose and placement of the magnetic material are also a factor in the configuration of the system and its components.

The presently described embodiments include a system and method incorporating, in at least one form, software for staging the tooth movement using, for example, such a magnet based orthodontic appliance, any of the devices described in the patent application entitled, “Magnet-Based Orthodontic Appliance,” having U.S. application Ser. No. 17/744,037, filed on May 13, 2022 (and published as U.S. Publication No. 2022/0361984A1), which is hereby incorporated by reference herein in its entirety and others, including, for example, other magnet-based orthodontic appliances. The presently described embodiments could also be used to create in-house aligners, virtual positioning of the brackets on models and fabrication of indirect bonding trays and fabrication of various orthodontic appliances. Also, as noted above, there is currently software available for dental segmentation and modeling. However, those software packages currently on the market suffer from various deficiencies that are addressed by the presently described embodiments.

In this regard, the presently described embodiments, in at least one example, take the form of a dedicated software program (e.g., instructions and/or code which are stored on at least one memory which, when executed by at least one processor, cause the processor and/or associated system to implement the method according to the presently described embodiments) with design features that allow segmentation and movement of teeth on the virtual model. The presently described embodiments, in at least one form, will be used to virtually segment teeth and simulate teeth movements, fabrication of the magnet-based appliance and clear aligners for orthodontic treatment. In at least one form, the software embodying the presently described embodiments will be accessible to technicians (e.g., full access with all possible editing features for tooth movement and fabrication of the appliance) and doctors (e.g., partial access using the features provided, they would be able to provide their requests and recommendations)

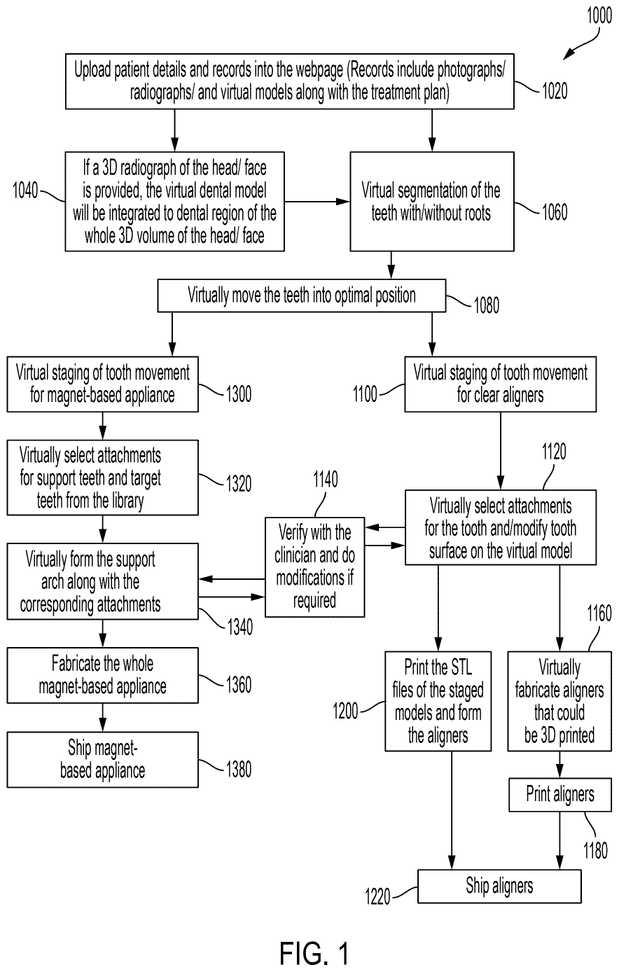

With reference to FIG. 1, a method 1000 according to the presently described embodiments is illustrated. As shown, the method 1000 is initiated by uploading patient details and records into a webpage (at 1020) to be utilized by the presently described embodiments.

These items of information could take a variety of forms including for example, photographs/radiographs/and virtual models along with the treatment plan. In at least one form, for example, a webpage may be provided with two access points for entering patients' details-one for the doctors (doctors' access is provided after they obtain the required training) and one for the patients (after they team up with a doctor to obtain treatment using the magnet-based appliance or other appliance or procedure). The contemplated web page may take a variety of acceptable forms and, for example, might also include details of the company/promotional/material/educational/material/shopping site for purchase of any material/owned/promoted/recommended by the company.

Next, if a 3D radiograph of the head/face is provided, the virtual dental model will be integrated to dental region of the whole 3D volume of the head/face (at 1040). This could be accomplished in a variety of suitable manners. At this point, virtual segmentation of the teeth with or without roots is completed (at 1060).

It will be appreciated that such segmentation may be accomplished in a variety of manners according to the presently described embodiments. Variations of these segmentation methods may be a function of factors such as, for example, the input data and the ultimate reason for completing the segmentation, e.g., generation of magnetic orthodontic appliances, aligners, or other appliances, execution of various dental procedures, . . . etc.

In at least one example, the presently described embodiments take the form of a software package that receives input from patients, technicians, clinicians, and doctors. As such, in one example, the software allows for a clinician to provide at least three (3) types of data:
Scenario 1: 3D virtual model of the teeth along with some part of the surrounding gingiva obtained by using a 3D intraoral scanner, along with 2D facial and intra-oral pictures and 2D radiographs.
Scenario 2: 3D virtual model of the teeth along with some part of the surrounding gingiva, 2D facial and intra-oral pictures, DICOM volumes of the head, including the upper and lower jaws and the teeth, obtained by a Cone Beam Computed Tomography (providing 3D radiographic images)
Scenario 3: 3D virtual model of the teeth along with some part of the surrounding gingiva, 2D intraoral pictures, 3D facial pictures, obtained with a 3D camera or any image capturing device that could capture the 3-dimensional images (and 3D radiographs obtained using Cone Beam Computed Tomography or any other 3D radiography.

Depending on the type of input, the presently described embodiments may be executed in different ways. As to the described example input scenarios, the following description provides example of actions that may be implemented.
Scenario 1: Only 3D intra oral scan is received.

Import the 3D intra-oral scan of the teeth (both arches) and the surrounding gingiva, software accepts the upper and lower models in occlusion.

### Labelling Tool:

Labelling the models—Label the models using a tool provided in this page as U for upper and L for lower. The tool opens the letters U and L which could be moved around using the mouse or arrow keys or using finger touch/stylus on a touch screen, once the letters are hovering over the individual models, hit enter. The letters won't be visible until the tab ‘Label’ is hit.

### Orientation Tool:

Tab named ‘Orientation’ on the software would be used to mark 5 lines on the models: four on the sides and one in the front to identify the 3D orientation of the arches with each other. Each line would be labelled individually as UR posterior line, UR anterior line, front line, UL anterior line and UL posterior line or using any other similar terminology. These lines would remain invisible until ‘orientation tab’ is hit.

Open ‘orientation’ tab, small tabs with the above-mentioned names will pop up-hit each Tab, a line will form-position the lines at the appropriate place with the mouse or using finger touch/stylus on a touch screen.

Lines on the sides: 1. From the mesio-buccal cusp of the upper first molar extending down to the lower arch based on the occlusion captured by the intra oral scan and verified by a 2D or 3D radiograph. 2. Line from the upper canine tip extending to the lower arch. In the absence of first molar, a line from mesio-buccal cusp of upper second molar could be used, if not a line from the buccal cusp tip of upper second premolar (if not upper first premolar) should be extended to the lower arch. In the absence of upper canine, a line from the upper first premolar cusp tip could be used. If the teeth are worn down and a cusp tip could not be identified, an arbitrary line could be made along the mid axis of the tooth in the mesio-distal plane.

Line at the front: Vertical line from the mesial edge of the upper right central incisor, (if not upper left central incisor), extending to the lower arch. If both the teeth are absent, mesial edge of the lateral incisor or any visible landmark in the front could be used.

Once the lines are marked, the software automatically identifies these lines and orient the upper and lower models into this original occlusion, any time the ‘orientation tab’ is hit.

### Numbering:

Number the tooth: Once each model is identified, each tooth in the arch should be assigned a number or a letter, the number involves two alphabets that represent the quadrant which the tooth belongs to and the number of the tooth in the quadrant starting from the front going backward. The quadrants would be named as UR (upper right), UL (upper left), LL (lower left), LR (lower right) with first tooth from the center as 1 and the last tooth as 8. So UR 7 means the 7th tooth from the front, in the upper right quadrant.

An animated arch would be there on the side and the numbers entered on each tooth would be immediately reflected in animated arch. If a tooth is missing, the number will be crossed out, so that the animated arch will not have that tooth.

How to do it? Hold the mouse over each tooth, hit enter—a box should open—type the tooth number—hit enter. Alternatively, the tooth could be identified using touch sense or stylus on a touch screen. In case of missing tooth, hold the mouse over the animated arch and hit delete, the corresponding tooth on the animated arch would vanish, then again hit enter-mark boundaries the record the space present in place of the missing tooth/teeth.

Software should have an option to substitute a tooth with a tooth adjacent to it, if the clinician plans to replace the missing tooth with the adjacent tooth, so that the space calculations and proportional ratio calculations are based on the planned outcome of the case. If that is planned, instead of deleting a tooth on animation, enter the number of the missing tooth on the next tooth and use a measurement tool to obtain the amount of space available.

### Segmentation Tool:

Each individual tooth should be separated into a separate object so that the 3D orientation of the individual tooth as well as its orientation in relation to the adjacent teeth and teeth from the opposing arch could be adjusted. Teeth are separated from the gingiva so that the crown can move. Boundaries of each tooth as well as its long axis should be identified, by the software or the technician. Once the boundaries are identified, software should be able to provide the teeth width, height and tooth proportion ratios such as Bolton's ratio, height to width ratio of the front teeth and proportion of the front teeth in relation to each other, like Golden ratio, recurring ratio etc. The 3D manipulation of all the individual tooth would allow the clinician to move the teeth into the planned final position virtually. It provides the amount of space that is needed to line up all the teeth in the final position or the amount of space that opens up if the teeth are moved in certain directions. Software also allows the automatic and/manual modification of the gingival architecture, teeth shape and position and width of the contact points between the teeth, to obtain optimal micro-esthetics. Micro esthetics include modification of the teeth shape including the height and width to obtain good proportions, adjustment of the proximal surfaces of the teeth to obtain good contact point between the adjacent teeth, adjustment of the gingival margins to obtain optimal esthetics.

Open ‘segmentation tab’—identify the model using the tabs provided here—tab 1—named upper model, tab 2 named lower model.

Tabs for aligning the model in the occlusal view, right view, left view and front view are provided, also, the holding the left key of the mouse down will allow free rotation of the model too. Also, there will be tabs on the left corner which allows 3D rotation around the axis of the model in all three planes of space. Additionally, the 3D orientation could also be changed by touch and slide actions using touch sense with or without a stylus.

Choose the model using the above-mentioned tabs-do the segmentation using the following tabs.
1. Boundaries-mesio-distal, cervical margins and vertical boundaries.

Mesio-distal boundaries: It is preferable to mark the points, viewing from the occlusal aspect, if a tooth is tipped, a proper view from the buccal, or lingual side would allow better orientation to mark the proximal surfaces. Marks are provided at the height of contour.

How to do it? Hit the tab ‘boundaries’, the model would automatically be oriented in the occlusal view, hit each tooth on the animation to identify the corresponding tooth on the model and mark the mesial and distal points. The points could be identified using a mouse on a traditional computer or with the help of a touch sense or stylus on a computer with touch screen interphase. The animation tab should immediately show the corresponding mesio-distal width, whereas the actual model would show two planes in line with the mesial and distal contacts of the tooth. The planes would also possess three dimensional arrows, in addition to circles in three directions to allow the change in orientation of the proximal limit planes. The plan is to get the proximal limit planes at the height of contour, where the maximum contact occurs and allows precise calculations of the width and space requirements in each dental arch.

Vertical boundaries: Additional tabs for locating the cusp tip of the teeth and the cervical margin, in order to identify the vertical boundaries of the teeth. Also, tabs will be provided for identifying the buccal grove of the upper and lower first molars and second molars. These are optional steps, software would still work without locating these landmarks.

How to do it? Hit the tab with a specific name, for example to identify mesiobuccal cusp tip to lower right first molar (LR6), hit the corresponding tip on the model, it would be positioned over the cusp tip. For the buccal grove, two points-one at the occlusal margin on the buccal surface and other at the middle third of the tooth would allow formation of a line.
2. Long axis of the tooth:

As soon as the mesial and distal boundaries of each tooth are completed, a line corresponding the long axis of the tooth pops up too, this long axis orientation of the tooth as identified by the software would be manually adjusted after looking at the tooth from the buccal/lingual view and the proximal view of the crown.

Any adjustments in the proximal limit planes or long axis could be accomplished by locating the corresponding points with the mouse—hit enter—and then readjust. Tabs would be provided for adjustment of the long axis of the tooth and mesial and distal proximal limit planes.

We can go back to each tooth, by using the animated arch on the side, as mentioned below too.

Measurements: (Optional tool for clinicians, or techs if they want to do any of these, if required)

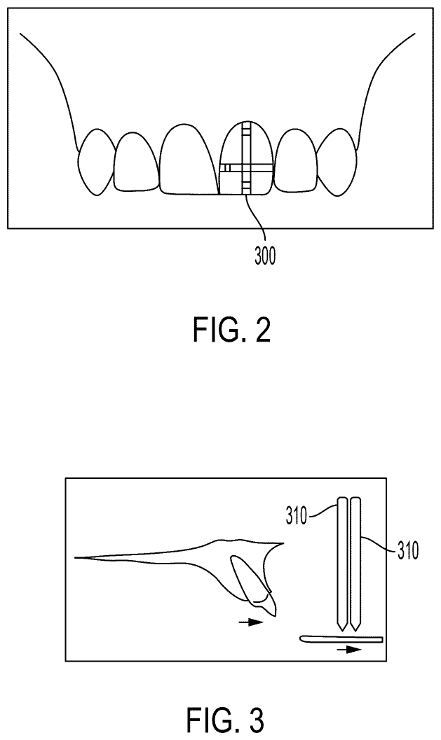

In at least one form, teeth and other items can be measured using a measuring tool taking the form of, for example, a symbol of a ruler or a picture, such as item 300 shown in FIG. 2. In this regard, a simple measurement tool is available to take 2D and 3D linear measurements and any 2D angular measurements. It allows, measurement of any space, extraction site, height of teeth, width of teeth, molar rotations, intermolar width etc.

After doing all these, hit tab ‘next’ at the lower right corner-multiple points would form at the cervical margin of each tooth, all around its periphery. These points are all connected to delineate the crown of the teeth from the gingival margins. Note: software recognizes the cervical margin based on the color difference on the scans or the discontinuity of the crown from the gingival margins. These points are adjustable by the technician so that the individual tooth could be segmented without any portion of the adjacent tooth/teeth. If technician is satisfied with the points around the tooth, hit ‘next’, we should be able to see the segmented crowns of all the teeth.

Using the animated arch, an individual tooth could be identified, use tab ‘isolate’ to isolate each segmented tooth so as to sculpt the proximal surface, if required. The segmented tooth most likely presents with flat proximal surfaces representing the plane cut, at times it might include the adjacent tooth surface too, if the proximal boundaries and hence the proximal limit planes were identified wrongly.

### Sculpting:

Use a sculpting tool (which could be adjusted using a millimeter scale, use only decimals of a millimeter), to smoothen the proximal surface, very minimally. Purpose is to ensure that each tooth is segmented with its true anatomy and no portion of the adjacent tooth forms a portion of this tooth (to avoid any potential mistake from the formation of a wrong proximal plane). ‘Add’ tool from the sculpting tool allows reshaping the individual segmented tooth to mimic restorations like broken edges, restorative build up to compensate for small sized teeth. Sculpt tool should have a library of all the teeth from which one could choose in order to replace any missing tooth or do quick and easy restorations of broken teeth.

How to do it?

Select the sculpt tool-there should be options for knife, brush (brush could be able to remove or add in tiny layers)—select the specific tool with the mouse—select ‘add/subtract’ (based on whether to remove or add to the surface' holding the left key down use mild brush strokes to remove or add. Alternatively, a stylus could be used over a touch screen for easy sculpting with paint strokes.

How to do restorations or replace missing teeth using the library?

After the planned teeth movement has been achieved, select the space or the broken or small sized tooth and hit delete, replace with one chosen from the library, by drag-place it in the right spot and hit enter. Alternatively, it could be done by identifying a segmented tooth and moving it into a trash bin using the mouse-cursor combination on a computer screen or using touch-drag combination on a touch pad.

Repeat all the steps on the second arch.

Tooth size-arch length discrepancy evaluation:

Once the proximal planes are located for each tooth, the tooth measurements show up on the tooth measurement tab.

Technician or clinician will use the ‘arch form’ tool and draw the expected arch form, based on the treatment plan provided by the doctor. For example, he would position the incisal edge of a central incisor as per the order and draw the arch form based on this. This arch form is expected to be at the point of contact of all the teeth in the final position. Software would calculate any space excess/space deficiency in the arch. A stylus over a touchscreen allows easy drawing of the arch form.

### Tooth Movement Tool:

Tooth movement tool is used to simulate the planned treatment.

How to do it?

Select each tooth from the animation arch. Use the tools for rotational and translational movement from the tabs. The movement tabs would be visible over the tooth when the tooth movement tool is chosen. The movements could be done either turning the wheels in the three planes of space, or using numbers in terms of degree and millimeters in along all three planes of space. Technician should have the freedom to select the center of rotation of each rotational movement. For e.g., a tooth could be virtually tipped lingually with the center of resistance at the root apex, so that the crown of the tooth will move lingually whereas the root apex stays at the same position. In another situation, the tooth could be tipped lingually with a center of rotation of the tooth located within the middle third of the root. In this situation the tooth will move with lingual tipping of the crown and labial movement of the root. In either case, the precise movement in millimeters and degrees should be displayed while the tooth is being moved. Also, software should show the amount of space that opens up, or the amount of space that is needed when a tooth or a set of teeth are being moved. A touch pad that is receptive to multi touch function, in combination with finger touch or a stylus facilitates easier 3D manipulation of the teeth in order to change their position and orientation in all three planes of space.
Scenario 2: Intra oral scan and CBCT are received.

The intra oral scans provide accurate representations of the teeth surface and the gingiva, but they do not show the root outlines. The CBCT volumes provide the near accurate representation of the teeth root, although it does not outline the gingival margins well.

In cases where a clinician wants to have a precise 3D orientation of the teeth including the roots, the software allows merging the CBCT scans with the intraoral scans so that when the teeth are segmented, they include the roots of the respective teeth as well. This merging of the intraoral scan with the CBCT allows the technician to obtain individual teeth with roots so that the whole tooth is moved in all three dimensions of place (instead of only the crown of the tooth, which is done in cases with most of the existing aligner systems, with the possible exception of very few systems). In all other appliance systems (in both clear aligners or traditional fixed appliances) this step might not be significant as the tooth movement predicted by the software is not replicated in the clinical situation due to inherent biomechanical limitations of the appliance systems in terms of root movement. The proposed magnet-based appliance is expected to overcome this shortcoming of the other appliance systems, hence segmentation of the whole tooth including the root gains significance.

How to do it?

Import the CBCT volume into the software. The whole CBCT volume can be segmented from the level of the basal bone (respective bones that hold the teeth) on each arch to the occlusal surface of the teeth, so that technician could open the respective regions of each arch side by side. The segmentation is done so that the whole occlusal surface of each of the arches could be obtained. Arches can be labelled as ‘upper arch’ and ‘lower arch’.

Segment the intraoral scan of each arch as one whole unit, few millimeters above the cervical margin of the tooth.

Use tool for ‘merging CBCT and intra-oral scan’, open the tool, open the segmented intraoral scan of one arch, open the segmented CBCT volume of the same arch—identify 5 points on the surface of the teeth on both intra oral scan and CBCT, so that the software will identify the respective points and get a best fit of the surfaces on the both of them. The 5 points could be anywhere on the teeth, at any position, but the technician should select the same points on both the intra oral scan and the CBCT volume, so that the software obtains a best fit. If there is any visible error while the merging takes place, technician should have the option of manually selecting the intra oral scan and orienting it over the CBCT volume using tabs for rotational as well as translational movements in all three planes of space. Merging is completed by using a ‘save’ tab.

Repeat the steps for the next arch.

Newly merged sections will look as if the crowns of the teeth are opaque, but the roots from the CBCT volume are transparent or opaque. The merged sections allow slicing in all three planes of space (similar to the slicing of the teeth on a 3D DICOM volume of a CBCT.

Select each tooth using the animated arch, since the teeth are already numbered, the cursor would be identifying the corresponding crown. Create proximal planes, segment the crown as mentioned previously-use the slice option and identify four points (mesial, distal, labial and lingual surfaces) at cervical margins of the teeth, nearly one third the distance from the cervical margin towards the root apex, middle of the root, one third from the root apex and identify the root apex. These points would be able to identify the root surface (absolute anatomy is not essential)—do adjustments as needed—Segment the tooth using the tab ‘segment the root’—number the root using the same numbering system. Use a tab ‘merge crown and root’, identify the corresponding crowns and roots and merge them. Identify the new long axis using the crown and the root orientation.

Center of rotation—A tab ‘center of rotation’ could be used to identify the center of rotation of the whole tooth.

Technician should be able to identify and modify the center of rotation of the entire tooth, which forms the geographic center (pivot) around which tooth will undergo any kind of rotational movement.
Scenario 3: Intra oral scan, CBCT volume and 3D facial picture, all three are received.

First two steps are completed as described above, except for a slight modification. The CBCT volumes are not segments at the level of the basal bone. The whole CBCT scan is merged with the intra oral scan, and the teeth (both the crown and root) are segmented.

Import the 3D picture, open the 3D picture and the merged volume (merge between intra oral scan and the CBCT volume) side by side. Match the soft tissue outline of the CBCT with the soft tissue outline of the 3D picture using a 5-7 target points (or areas). The technician should be able to identify corresponding points—using the tool ‘merge CBCT and 3D picture’ so as to merge both. There should be an option for manually adjusting the two (if required) to get the best fit.

The merged 3D picture should be transparent so that the hard tissue structures inside (including the 3D CBCT volume and the intra oral scan) would project through the transparent 3D picture allowing the technician to plan the dental movements. The 3D pictures are obtained in closed mouth position, and one in smile position. This allows treatment planning in terms of incisor position in relation to the upper lip.

As a possibility, in at least one form of the presently described embodiments, AI can predict the soft tissue changes as and when a technician moves the upper incisors forward or backward by bodily movement or tipping.

In addition, as indicated above, currently available segmentation and/or modeling software has many deficiencies. Accordingly, the presently described embodiments, e.g., software embodying the presently described embodiments, uses, in at least one form, a touch screen interface and/or a stylus pen that helps to define the boundaries of tooth/teeth/or segments of jaw and plan orthodontic and orthognathic surgical treatment. It could also be used to design/plan restorations and dentures as well as dental implants. It could be used to design various orthodontic appliances like magnet base orthodontic appliance. An alternate way could be to use a finger touch and/swipe to identify the jaw and/or tooth movements in the desired direction and orientation.

In another modification, a camera could capture hand/wrist or finger movements to simulate the movement of the teeth/jaw or restorations, once a jaw segment and/or tooth is identified with a mouse, stylus or finger touch.

In another modification, voice control could be used to identify a jaw segment and/or tooth and plan the movement in the desired direction and orientation.

### Touchscreen

The touchscreen could be a personal computer/laptop/tablet with a touchscreen function which allows the software to be manipulated by touch movements using a fingertip and/or a stylus. The touch screen used would be receptive to single touch or multi-touch command. It could also be personal computer/laptop/tablet with a front facing camera which captures hand/wrist/finger movements which could mimic the planned treatment simulations like translational and rotational movements in all three planes of space. It could also be a mere touchpad, without a monitor. But this touch pad allows the touch movements using fingertip and/or a stylus to be mimicked on the monitor of a standard PC which does not have the touch function. This touch pad could be an independent touch pad dedicated for this software or a computer keyboard with a touch pad as a part of it but modified for the specific software to work. Independent touch pad or a keyboard with a touch pad would be connected to the PC with a USB cable, Wi-Fi connection or Bluetooth. Any of these devices which are not always connected to a power outlet would be powered by a battery of appropriate capacity.

### Stylus

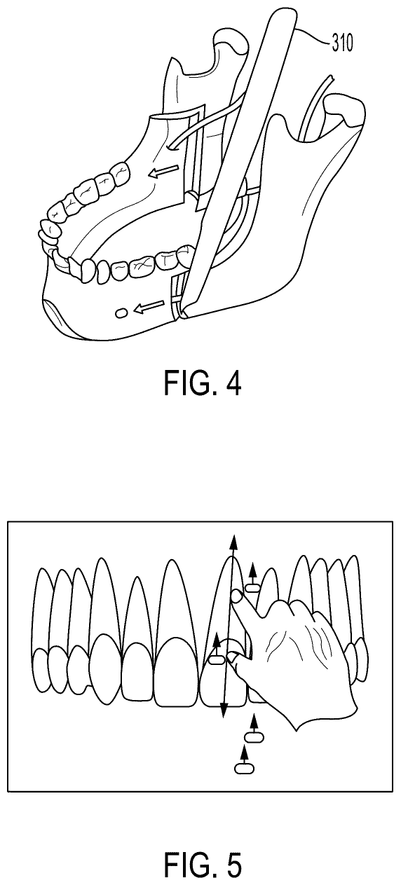

Referring to FIG. 3, a stylus 310 (shown in two positions in FIG. 3) that can be used according to the presently described embodiments, is a standard stylus which only facilitates easy definition of the boundaries, or ease of drawing the shape of a restoration. In such cases, stylus merely replaces a cursor or pointer, like while using a standard mouse of a computer. This stylus could be used to design the virtual appliances like dentures, removable orthodontic appliances, implants and other prosthesis including maxillofacial prosthesis. The movement of a stylus over a preprogrammed region of the touch screen or a touch pad in a linear or circular motion could simulate a pre-planned tooth movement or movement of a segment of jaw. The stylus could be dragged over the touchscreen or touchpad to mimic the movement. The stylus could be used to select ‘attachments’ from a library of attachments with various shapes and sizes, and drag and attach the ‘attachments’ on the tooth surface, the stylus allows easy manipulation of the direction of the attachments, formation of various shapes and sized according to clinician's preferences. Additionally, as shown in FIG. 4, a stylus 310 could be used to plan surgical cuts on the jaw segments and the movement of the stylus could be used to mimic the planned surgical movements of the jaw segment.

The stylus could also be made to touch a specific region of the touch screen programmed specifically for this function before doing the translational and rotational movements. Provision would be made on the software to facilitate the use of configurations like tooth or a simple rectangular bar which could be moved around after identifying the tooth that needs to be moved. The tooth which is being rotated or the implant which is being planned could be identified initially using the touch function.

In cases with regular PC monitors or laptops without a touch screen interface, a modified keypad with a touch pad interface could be used. Or regular computer mouse could be used as a pointer to identify the object (in this case, it could be a tooth, implant or a restoration)

Provision would be provided on the touchscreen to use the stylus to orient the whole dental model in all three planes of space.

The amount of movement of the stylus would be calibrated at 1:1 or 1:2 ratios or as programmed in order to reduce the sensitivity of the software in mimicking the movement of the stylus. For example, a 5-degree rotation of the stylus could move the virtual object by 5 degree or 2.5 degree as has been set by the software. Provision could be made in the software to adjust these settings by the operator.

Stylus could be used to draw the shape of the dental arch which would function as the template to form the arch form and estimate the access/deficiency of dimensions of the tooth that could fit into that specific arch shape. Drawing could be done against the backdrop of a transparent grid formed over the existing dental model.

Or stylus could be used to modify the dots that connect an arch form, in order to make minor, but precise adjustments to the shape of the arch and the position of the jaw segment and/or tooth.

The stylus could be used to draw the shape and outline of a planned orthodontic appliance like magnet based orthodontic appliance. It could also be used to drag and place braces for the fabrication of indirect bonding trays. It could also be used to design the indirect bonding tray.

### Using Fingers on the Touch Pad

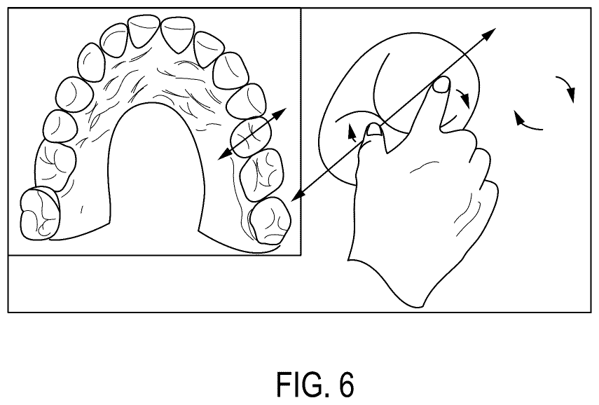

With reference to FIGS. 5 and 6, finger touch and swipes could be used to move the whole dental model and rotate and orient the model in all three planes of space. Provision would be provided to obtain a true occlusal, frontal, right lateral, left lateral and posterior views with the help of tabs designated for the same.

Once the segmentation has been done with/without the help of a stylus, finger touch over the touch pad could be used for virtual tooth movement. Designated area could be used to swipe the finger/fingers in the right direction in a linear (e.g., FIG. 5) or angular (e.g., FIG. 6) fashion to obtain virtual tooth movement.

Hand/Wrist/Finger Swipes and/or Gestures

In another modification, a camera on the touchscreen could recognize the movements of a person's hand/wrist/palm/fingers in all three planes of space and the software could capture these movements to simulate the virtual movements of the object (in this case, teeth or implants or jaw segments) on the monitor. Camera could be at the periphery of the touch screen or it could be under the screen. The camera and hence the software could be trained to identify the outer and the inner aspect of the palm to replicate the outer and the inner surfaces of the teeth, so that the movements and the orientation of the palm could be replicated by the virtual teeth/implant on the PC or touch screen. Specific gestures could be used to identify and place various attachments over the tooth surface. Gestures could be used to turn the virtual model on the monitor and/or move the teeth in all three planes of space. hand/finger swipes on the touch screen and hand/finger gestures in front of the touch screen would be calibrated to provide a specific amount of movement of the tooth or jaw segment for the amount of movement by the hand/finger swipe or hand/finger gesture. Swipe over the touchscreen while touching the screen could be controlled as the swipe could be continued until the desired amount of change has occurred. Gestures could be aimed to provide a specific amount of movement. For example, after identifying a tooth by touch or voice command, a gesture across the screen from left to right without touching the screen would move that tooth to right side by 0.5 mm, so that two swipes are required to move the teeth by 1 mm. In a similar way, if the rotational movement is initiated, a rotational gesture by the palm would rotate the tooth or segment by 1 degree.

Voice control of the software:

The software would be programmed to function with the help of voice control which could be used to identify individual arches, individual teeth, align, level, rotate and change in the mesio-distal angulation as well as labiolingual position and inclination. Software would require initial calibration and voice recognition.

### Steps:

Identify the arch—Maxilla/Mandible either calling out ‘maxilla’ and ‘mandible’ or using codes for each arch, like ‘max/upper’ for maxilla, ‘lower’ for mandible.
Orient the models—By calling out ‘Frontal view’, ‘Right lateral view’, ‘Left lateral view’, ‘Occlusal view’
Develop the archform—Form the arch along the templates provided, each archform could be identified with a name or code. For example-tapered archform would be called as ‘tapered’ or ‘T’.
Identify each tooth: By calling out the tooth number or a code designated for each tooth.
Tooth movements: After orienting the models in the appropriate view, and identifying the tooth, movements could be initiated by calling out ‘buccal/labial translation’ ‘mesial translation’. Amount will be determined by saying ‘2’ or ‘3’, and default unit would be in millimeters. Additional movements like, ‘distal root tip’, ‘3’would provide a distal root tip of 3 degree, as the default unit for distal root tip would be in degree. Also, ‘torque’ would indicate, lingual root movement. Center of rotation of each of these movements would be set in the software. Derotation would be done by orienting the model in the occlusal view, followed by ‘identification of the tooth’ and saying ‘clockwise/counter clockwise’ followed by ‘3’ or whatever be the amount of rotation in degrees.
Attachments: Voice control could be used to select and place an attachment over the tooth surface, with a default being the center of the crown of the tooth. Other commands could be used to change the size and orientation of the attachments.

### ‘Replicate’ Function of the Software

The software would have the capability to orient contra lateral tooth of any of the incisors in one dental arch in the same labio-lingual inclination and vertical position using a tab ‘replicate’. It allows the teeth from canine posteriorly to obtain similar mesio-distal angulation and vertical position, as well.

‘Mirror/negative’ function of the software

The software would have the capability to orient contra lateral tooth of any of the incisors in one dental arch as a mirror image in the mesio distal plane using a tab ‘mirror/negative’. It allows the teeth from canine posteriorly to obtain a mirroring of the labio-lingual inclination, as well.

All the above-mentioned options would have a limit for the segment of jaw and/or tooth movement based on a ‘collusion’ with an adjacent segment of jaw and/or tooth. An override function could be used to avoid the collusion, if the clinician wishes to do so.

A software with a touchscreen interface which could be operated with a stylus pen and/finger swipes which enables the operator to mark the boundaries of the tooth, restoration or segments of jaw draw the design of the appliances and virtually plan the movements of the tooth, implants or segments of jaw would be more precise, user friendly and efficient than the currently available software for the same. The gesture control and voice control would facilitate better infection control in case of multi-user scenarios.

Referring back to FIG. 1, after the virtual segmentation is completed (at 1060), a user can virtually move the teeth into optimal position (at 1080). It will be appreciated that such movement may be accomplished in a variety of manners and, in at least one form, depends on whether aligners or magnetic appliances, or both, are being generated for a patient. It will further be appreciated that the software implementing the method according to the presently described embodiments may include instructions or code to cause the system to allow for generation of aligners, magnetic appliances, or both. In this regard, for example, the entire method 1000 or selected parts may be implemented according to the presently described embodiments. For example, the instructions or code may be configured to cause the system to allow for generation of only aligners, or only magnetic appliances, or various combinations of the noted features in accord with implementation of the presently described embodiments.

In the case of generation of aligners and corresponding virtual staging of tooth movement for such aligners (at 1100), attachments for the tooth are virtually selected and, if necessary, modification are done on the tooth surface on the virtual model (at 1120). This may be accomplished in a variety of manners, but in at least one form of the presently described embodiments, such selections are verified with the clinician and modifications are completed, if required (at 1140). In one form, STL (e.g., standard tessellation language) files, or other files (e.g., Wavefront Object file or OBJ file, 3D Manufacturing File or 3MF file, Additive Manufacturing File format or AMF file), of the staged models are printed, and aligners are formed (at 1200). In another form, aligners are virtually fabricated for 3D printing (at 1160) and then printed (at 1180). It should be appreciated that although the example fabrication system may take the form of a 3-dimensional printer, other systems to form the generated aligners may be used (such as, for example, suitable molding, casting, extrusion, roll forming, forging, or stamping systems). Various output techniques may be realized for achieving these objectives. In either case, the aligners are then shipped (at 1220).

With continuing reference to FIG. 1, in the case of generation of magnetic-based appliances and corresponding virtual staging of tooth movement for such appliances (at 1300), attachments for the support teeth and target teeth are virtually selected (at 1320). Then, support arches, along with corresponding attachments, are virtually formed (at 1340). This may be accomplished in a variety of manners, but in at least one form of the presently described embodiments, such selections are verified with the clinician and modifications are completed, if required (at 1140). It should be appreciated that the general term “attachment” as used in connection herein with the magnet-based orthodontic appliance refers to various attachment elements useful for implementing the magnet-based orthodontic appliances and described, for example, in the patent application entitled, “Magnet-Based Orthodontic Appliance,” having U.S. application Ser. No. 17/744,037, filed on May 13, 2022, which is hereby incorporated by reference herein in its entirety. In this regard, for example, attachments for support teeth take the form of and are also referred to as attachment magnets. Attachments for the target teeth, for example, take the form of and are also referred to as target magnets. Attachments for the support arches of the magnet based orthodontic appliances, for example, take the form of and are referred to as mounting magnets if they correspond to attachment magnets on support teeth and take the form of and are referred to as therapeutic magnets if they correspond to target magnets on target teeth.

Then, the magnetic based appliances may be fabricated (at 1360) and shipped (at 1380). It should be appreciated that the fabrication system may take any form suitable for a particular application. For example, the system may take the form of a 3-dimensional printer or other system to form the generated magnet based appliances (such as, for example, suitable molding, casting, extrusion, roll forming, forging, or stamping systems). It should be appreciated that accommodations for forming (including separately forming) the magnetic material, integrally forming the magnetic material into or as the support arch, and/or attaching magnetic material to the support arches, will be made and suitable actions taken. Various output techniques may be realized for achieving these objectives.

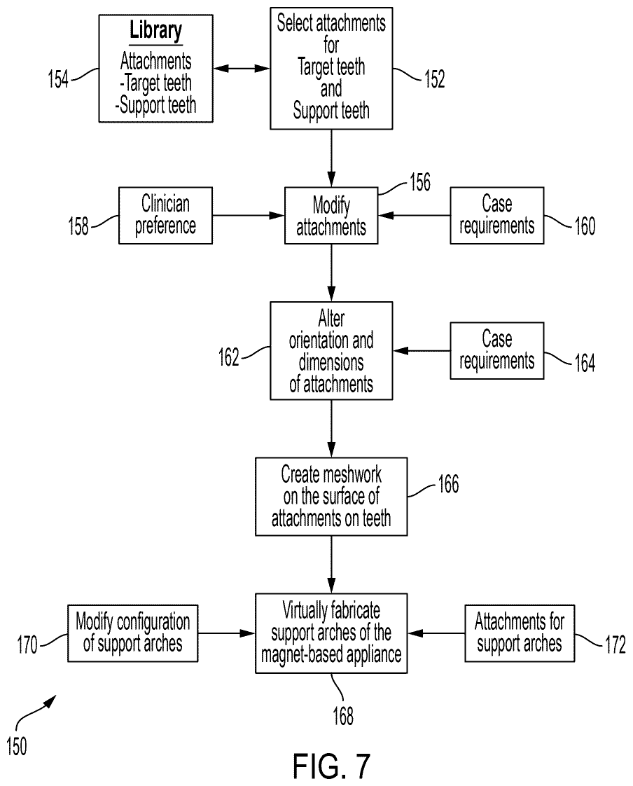

As noted, items 1320 and 1340 (and 1140) may be accomplished in a variety of manners. However, in at least one form, a method or technique 150 may be implemented as but one example of these items of the presently described embodiments. In this regard, with reference to FIG. 7, the method 150 includes selecting attachments (e.g., attachment magnets and target magnets) for target and support teeth (at 152). These attachments, in one form of the presently described embodiments, may be retrieved from a library (at 154) or other storage medium. Next, the attachments may be modified, if needed (at 156). In at least some forms, the target attachments tend to require more modification than the support attachments. Also, any such modifications may be accomplished based on the clinician's preference (at 158) or case requirements (at 160). The orientation of the attachments may also be altered, if necessary (at 162). This may be accomplished in a variety of manners but, in one form, is based on the case requirements (at 164). A meshwork may then be created on the surface of the attachments to form a negative replica of the tooth surface (at 166). This is accomplished to allow bonding, e.g., improved bonding, of the attachments to the tooth surface with, for example, suitable resin.

Also, the support arches of the magnetic based appliance are then virtually fabricated (at 168). In at least one form, a default support arch is provided for the virtual fabrication. If needed, the default arch may be modified (at 170). Also, attachments (e.g., mounting magnets and therapeutic magnets) are provided to the support arches (at 172). Again, this may be accomplished in a variety of manners. However, in at least one form, attachments are formed on the default support arch based on matching magnets of target teeth. Default magnets may also be based on clinician's desired movements. It will be appreciated that support magnets will usually differ from target magnets. All attachments, or magnets, may be modified by clinician.

To further describe the magnetic based appliance fabrication, once the teeth segmentation and subsequent planned teeth movement is completed, the software would be used to fabricate a virtual prototype of the appliance. Software would have default prototypes which includes the support arch and the various attachments. The technician should be able to change the design of the support arch and the various attachments, alter the shape and size of everything based on the case requirements and the staging of the teeth movement.

How to Do it?

By selecting the tool ‘Form aligner’, a default option should allow the software to form an initial prototype of the appliance based. The initial prototype is made based on the arch shape, pre-planned size of the attachments on the teeth (based on the average size of the teeth in both vertical and mesiodistal direction) and the support arch with a distance of 0.5 to 1.0 mm or anything else that was predetermined for a specific case, between the attachments on the teeth and the attachments on the support arch.

### Attachments on the Teeth:

Attachments (e.g., attachment magnets) on the support teeth (called support teeth in the appliance): Default attachment for the support teeth in both arches would present with certain features that allow a rest or extension from the support arch to occupy a recess or a rest seat, it allows the rest or extension to be securely locked in place. The size and location of these attachments would be set for a specific dimension, but the technician would have the freedom to alter these using a tool bar that allows change in dimensions or using a tool with millimeter features in x, y and z planes. The surface of these attachments that face the tooth should precisely match the contour of the respective tooth, also, they should form a mesh at the base automatically, in order for the resin cement to form a firm bond with the tooth surface.

Attachments on the target teeth: By default, the attachments (e.g., target magnets) on the target teeth should be formed to certain specific dimensions, default would be rectangular or square blocks of specified dimensions depending on the type of teeth. For example, lower incisors would have a rectangular block of 4 mm width and 6 mm height whereas upper incisor should have a width of 6 mm and height of 6 mm. By default, the thickness of the block would be at least 1 mm at the area of minimum thickness. This allows the formation of a square/rectangular block of 1 mm thickness in addition to the thickness needed to form a firm adaptation to match the contour of the tooth surface. So, software should automatically form the blocks at appropriate sites followed by addition of the rest of the contouring to match the tooth surface. The surface of these attachments that face the tooth should precisely match the contour of the respective tooth, also, they should form a mesh at the base automatically, in order for the resin cement to form a firm bond with the tooth surface.

In another variation, there could be a contoured base on the tooth and the attachment, or the block might be larger or smaller in dimension than the contoured base, it could be even extending beyond the surface of the tooth.

In a third variation, the attachment on the target tooth could be a hollowed-out shell, which would receive a square or rectangular block.

Default version would allow the formation of the support arch on the middle to cervical region of the tooth on the lingual side. Modifications are possible.

The technician should be able to adjust the dimensions in all three planes, as and when required. Also, all the various attachments are available in a library within the software.

### Attachments on the Support Arch:

The attachments corresponding to the support teeth (e.g., mounting magnets) may or may not match the dimensions of the attachments on the support teeth. Default version would have an extension arm/rest that precisely fits into the rest seat on the attachment on the support teeth. A variation would be to have extensions from the support arch that fits into the rest seat. Other modifications would be possible too. For example, attachments on the support teeth and the corresponding attachments on the support arch would be in close approximation. In such a case, there could be some projections on either of the approximating surfaces.

Attachments corresponding to those on the target teeth (e.g., therapeutic magnets) would precisely match the height and width of the attachments on the target teeth, default thickness would be 1 mm. But modifications are possible.

The surface of these attachments that face the tooth should precisely match the contour of the respective tooth, also, they should form a mesh at the base automatically, in order for the resin cement to form a firm bond with the tooth surface.

Support arch: The default version forms a support arch of a certain cross-sectional shape, preferable oval in cross section with the larger dimension in the horizonal plane. The default support arch would be formed based on the original arch form, but on the lingual side of the teeth adjusting to the thickness of the attachments on the teeth, 05-1.0 mm space and the thickness of the attachments on the support arch. Technician should be able to modify the shape and position of the support arch by using a ‘drag and draw’ tool, which allows formation of multiple points along the curved support arch. By dragging these points, the technician should be able to change the position of the support arch.

Although the tool ‘Form aligner’ would form a virtual prototype, the tabs with in the ‘form aligner’ would include, ‘attachments on teeth’, ‘attachments on support arch’ and the ‘support arch’. A library would be available to drag and replace any of the attachments. By holding the mouse on any of these and hitting enter, would identify that specific component, which could be altered using various tools. Or, alternatively, a stylus or fingertip could be used to identify the attachments from the library and later dragged to replace the attachments already formed using the “Form aligner”tool.

Also, software should be capable of preventing any interferences that could potentially occur between various attachments while teeth are being moved, whether it is in the same ach or across the arches.

Software should be suggesting the use of bite planes, anterior or posterior, if inter-arch interferences could not be avoided. Also, software should allow display of occlusal contacts and tightness of the contacts using color mapping or in decimals of a millimeter.

Software will be able to alter the configuration of the tooth surface so that an aligner fabricated on the newly altered tooth model could apply differential pressure based on the indentations created on the individual tooth surface.

### Photograph Analysis:

Symmetry could be evaluated by creating a composite photograph using the mirror image of one side added to the other. Also, it should allow locating some points on the surface of the 3D picture, which would display measurements from the midline in both vertical and horizontal planes.

### Indirect Bonding Tray Fabrication:

Software should have a feature to fabricate an indirect bonding tray that could be 3D printed, in order to obtain precision in bonding of the attachments, as in the virtual model. It should also have a library that contains STL files of the commonly used brackets. The software allows virtual positioning of the orthodontic brackets and formation of an indirect bonding tray. The software automatically places the bracket on a pre-determined spot on the crown of the tooth. The technician or the doctor would have the freedom to do adjustments.

### Fabrication of a Guide for the Placement of the TAD (Temporary Anchorage Device):

Software should allow designing of a TAD guide, so that coupling of the TAD to the magnet based orthodontic appliance is possible, as has been precisely planned. It also facilitates the fabrication of a template for guiding gingivectomy.

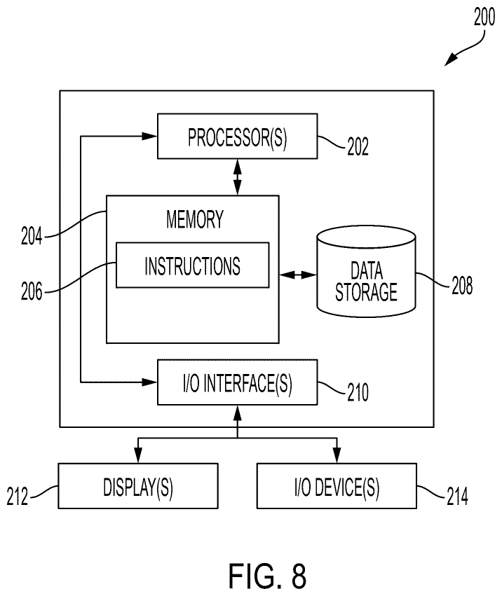

Referring now to FIG. 8, an example system 200 is shown into which the presently described embodiments may be incorporated. It should be appreciated that the system 200 is merely an example—it may take a variety of forms. For example, the above-described methods and/or techniques can be implemented on a system such as system 200 using well-known computer processors, memory units, storage devices, computer software, and other components. As shown in the example representation of such a system, the system 200 includes at least one processor 202, which controls the overall operation of the system 200 by executing computer program instructions 206 which define such operation. The computer program instructions may be stored in at least one storage device or memory 204 (e.g., a magnetic disk or any other suitable non-transitory computer readable medium or memory device) and loaded into another memory (not shown) (e.g., a magnetic disk or any other suitable non-transitory computer readable medium or memory device), or another segment of memory 204, when execution of the computer program instructions is desired. Thus, the steps of the methods described herein (such as, for example, method 100 of FIG. 1 and FIG. 7) may be defined by the computer program instructions 206 stored in the memory 204 and controlled by the processor 202 executing the computer program instructions 206.

The system 200 may include one or more Input/Output (I/O) interfaces 210 for communicating with other devices such as displays 212 and/or input/output (I/O) devices 214 via a network. The I/O devices 214 of the system 200 may also include a user interface that enables user interaction with various other elements of the system. The user interface may include I/O devices (e.g., keyboard, mouse, speakers, buttons, etc.) to allow the user to interact with the computer.

According to various embodiments, FIG. 8 is an example representation of possible components of a system including a processor for illustrative purposes. Of course, the system may include other components. Also, the system 200 is illustrated as primarily a single device or system. However, the system 200 may be implemented as more than one device or system and, in some forms, may be a distributed system with components or functions suitably distributed in, for example, a network or in various locations.

It will be appreciated that at least one form of the presently described embodiments will include an output interface to transmit the results of the presently described embodiments to a system (e.g., as represented generally by at least one of the I/O devices 214) to form or manufacture products embodying and/or manifesting the generated virtual design. Such a system may take any form suitable for a particular application. For example, the system may take the form of a 3-dimensional printer or other system to form the generated aligners or magnet based appliances (such as, for example, suitable molding, casting, extrusion, roll forming, forging, or stamping systems). If the system forms magnet based appliances, it should be appreciated that accommodations for forming and/or attaching magnetic material to the support arches will be implemented. Various output techniques may be realized for achieving these objectives.

Also, it will be appreciated that the structures and procedures shown above are only a representative example of embodiments that can be used to facilitate embodiments described above. In this regard, the various embodiments described in the examples above may be implemented using any suitable circuitry, hardware, and/or software modules that interact to provide particular results. One of skill in the computing arts can readily implement such described functionality, either at a modular level or as a whole, using knowledge generally known in the art. For example, the flowcharts illustrated herein may be used to create computer-readable instructions/code for execution by a processor. Such instructions may be stored on a non-transitory computer-readable medium and transferred to, for example, the processor for execution as is known in the art.

The presently described embodiments may be implemented using a variety of different interfaces for patient and clinicians, as well as manufactures of the aligners and/or magnetic based appliances. Examples of a form of webpages providing at least a portion of such interfaces are described below:
Webpage
Type—www.muorthodontics.com
Opens the page—
Inside the page—We would have tabs for the following.
Overview of the company with vision and a mission statement.
Doctors' page
Patients' page
Are you a candidate for magnet based orthodontic appliance?
How it works and how it's different/promotional material?
Educational material
Resources/products/shopping site
Support
Overview of the company with mission statement.
Doctors page: Register/Sign in
Register:
Name/Business address/Phone numbers
Home/Business/Personal
Proof of orthodontic training/Proof of magnet based orthodontic appliance software training
Submit—link send to email or passcode send to text/email. Once they receive the link/code, they can re-visit the site and form a sign in ID preferably their email and form a strong password.

An option is to have two step logs in too—down the line, because of security threats to patient's personal information including photos.
Sign in:
Login page will open by:
Going to www.muorthodontics.com and clicking “doctor login”
Going directly to www.muorthodontics/doctorsite.com
Login page will have a “Welcome to the magnet based orthodontic appliance Doctor Site”.
Under that, there will be space for credential to log in.
Username:
Password:
Log in
Forgot Login using an ID and a password
Once signed in—it goes into doctors account which has all the patients'records.
Contraindications for the use of magnet based orthodontic appliance should be present as a pop-up prompt, that the clinician has to accept and continue, if they wish to submit the case.
Layout once signed in, is as follows.
Details would be listed out as in web page.
Patients' details, treatment type, single arch/dual arch
Limited treatment/Full treatment
Magnet based orthodontic appliance only or magnet based orthodontic appliance with active clear aligners.
Type in doctor's treatment objectives
Upload the following.
Patients' photos
Radiographs—panogram (Mandatory), lateral cephalogram, CBCT and 3D facial scan (optional)
Intra-oral scan,
Treatment plan with staging/sequencing as requests, with a mention about the teeth that need to be moved and the teeth that need to be left as such.

Following details should be provided regarding the case.

The clinician should be able to provide instruction as to where they want to keep the incisors (in cases of generalized spacing or extraction). Based on the case requirement, space closure will be planned. In some cases, the entire space will be closed by taking the incisors back, whereas in some cases the space will be closed by backward movement of incisors and the forward movement of the posterior teeth. Clinician should also provide details of the kind of tooth movement that they wish to attain while moving the incisors back; instructions should be provided for tipping kind of movement vs translation kind of movement.

Indicate if they wish to use TADs for anchorage and the site of preferred TAD placement.

Submit the case, submission is allowed only after all the required material is submitted.

At this point the case is submitted. The relevant material is imported into the software and the treatment will be planned as mentioned below.

Once the virtual aligner and the staged teeth movement are ready, everything is sent back to the clinician who opens the ‘check magnet based orthodontic appliance’ application on the webpage, look for the patient's ID and verify whether the staged teeth movements meet their treatment requirements. Clinicians can ask for modifications and/or edit and modify the existing virtual model to their preferences and resubmit the case. The technician would reevaluate the feasibility of these modifications and do the best fit and return it to the clinician. Once the clinician accepts the planned movements, they should ‘approve’ the case and they will be charged automatically for the full case.

The software will be enabled to use artificial intelligence (AI) to identify the patterns used by clinicians and technicians while treating similar cases which allows the software to establish a treatment plan, reducing the workload on the clinician and the technician working on the case.
Patient's page:
Register/Sign in
Register using the ID provided by the doctor and a password created by the patient. They do not have any edit options on the webpage, they can watch the simulated tooth movement from start to finish.
Login page will open by:
Going to www.muorthodontics.com and clicking “patient login”
Login page will have a “Welcome to the magnet based orthodontic appliance Patient Site”.
Under that, there will be space for credential to log in.
Username:
Password:
Log in
Forgot Login using an ID and a password
Are you a candidate for magnet based orthodontic appliance?
Smile assessment questionnaire and a portal for uploading facial and intra oral pictures.
How it works and how it's different/Promotional material:
Explains how magnet based orthodontic appliance is different and superior to all existing appliance systems.
New materials/products launch, new events etc.
Educational material:
Who are we?
Links to our articles, eBook and other relevant material
Educational material specific to maligners
General material on biomechanics and how magnet based orthodontic appliance biomechanics could be better according to the current literature
Research on magnet based orthodontic appliance-summary of the papers and links would be uploaded
Links to CEs and promotional events on magnet based orthodontic appliance Links to general orthodontic CEs that will be conducted
Resources/products/shopping site.
All the available products would be available for purchase . . .
Attachments of various shapes and sizes, TADs specific for magnet based orthodontic appliance, modifiable TAD heads for TADs from other companies, debonding plier, (they can buy and stock it from our store)
Shopping cart
Support
Customer care service set up
Phone call, email set up to be dealt by IT support staff.
Note: “Check magnet based orthodontic appliance” could be the app, which could be on android and IOS phones where in patients can accept/modify the final outcome.

The exemplary embodiment has been described with reference to the preferred embodiments. Obviously, modifications and alterations will occur to others upon reading and understanding the preceding detailed description. It is intended that the exemplary embodiment be construed as including all such modifications and alterations insofar as they come within the scope of the appended claims or the equivalents thereof.

1. A system for generating a magnetic based orthodontic appliance comprising:
at least one processor;
at least one memory having code or instructions stored thereon that, when executed by the at least one processor, causes the system to:
receive information on a patent's teeth including at least one of photographic images, radiographs, virtual models, and a treatment plan for generating the magnet based orthodontic appliance;
virtually segment the teeth;
virtually determine attachments for target teeth and support teeth;
selectively virtually modify the attachments;
selectively virtually alter the orientation of the attachments;
create a virtual meshwork on surfaces of the attachments;
virtually fabricate support arches with corresponding virtual attachments;
output a virtual magnetic based orthodontic appliance.

2. The system as set forth in claim 1, wherein the attachments for target teeth are magnets or paramagnets.

3. The system as set forth in claim 1, wherein the attachments for support teeth are magnets or paramagnets.

4. The system as set forth in claim 1, wherein attachments for support arches are magnets or paramagnets.

5. A method for generating a magnetic based orthodontic appliance comprising:
virtually segmenting the teeth;
virtually determining attachments for target teeth and support teeth;
selectively virtually modifying the attachments;
selectively virtually altering the orientation of the attachments;
creating a virtual meshwork on surfaces of the attachments;
virtually fabricating support arches with corresponding virtual attachments;
outputting a virtual magnetic based orthodontic appliance.

6. The method as set forth in claim 5, wherein the attachments for target teeth are magnets or paramagnets.

7. The method as set forth in claim 5, wherein the attachments for support teeth are magnets or paramagnets.

8. The method as set forth in claim 5, wherein attachments for support arches are magnets or paramagnets.

9. A system for segmenting teeth, simulating teeth movement and/or generating an orthodontic appliance comprising:
at least one processor;
at least one memory having code or instructions stored thereon that, when executed by the at least one processor, cause the system to:
receive information on a patient's teeth including at least one of photographic images, radiographs, virtual models, and a treatment plan for the patient's teeth; and
virtually segment the teeth using a touch screen that accepts single touch or multi touch commands with finger touch on the touch screen, a stylus on the touch screen, gestures or voice control to virtually move the teeth.

10. The system as set forth in claim 9, further comprising:
virtually fabricating an orthodontic appliance.

11. The system as set forth in claim 10, wherein the orthodontic appliance is at least one of: an aligner or a magnetic based orthodontic appliance.

12. A method for segmenting teeth and/or generating an orthodontic appliance comprising:
receiving information on a patient's teeth including at least one of photographic images, radiographs, virtual models, and a treatment plan for the patient's teeth; and
virtually segmenting the teeth using a touch screen that accepts single touch or multi touch commands with finger touch on the touch screen, a stylus on the touch screen, gestures or voice control to virtually move the teeth.

13. The method as set forth in claim 12, further comprising fabricating an orthodontic appliance.

14. The method as set forth in claim 13, wherein the orthodontic appliance is at least: an aligner or a magnetic based orthodontic appliance.
